SEQUENCE LISTING (1 sequence)

SEQ ID NO: 1 - protein, 32 aa, Artificial sequence
  Synthesized
YLCAGRNDCIIAIKFEEKTAQHAAIENVFRLE

(19) United States
(12) Patent Application Publication
(10) Pub. No.: US 2006/0100147 A1
(43) Pub. Date: May 11, 2006

(54) Anticancer peptide

(76) Inventors: Edward Bradbridge Thompson, Galveston, TX (US); Aaron Loy Miller, Galveston, TX (US); Betty H. Johnson, Beaumont, TX (US)

Correspondence Address: GORDON & REES LLP, 101 WEST BROADWAY, SUITE 1600, SAN DIEGO, CA, 92101, US

(21) Appl. No.: 11/230,952

(22) Filed: Sep. 19, 2005

Related U.S. Application Data
(60) Provisional application No. 60/611,356, filed on Sep. 20, 2004.

(51) Int. Cl. A61K 38/17; C07K 14/82

(52) U.S. Cl. 514/12; 530/350

(57) ABSTRACT

A polypeptide and methods of using the polypeptide for treating malignancy by administering to a subject a composition of the polypeptide. Pharmaceutical compositions of the polypeptide.

### BACKGROUND OF THE INVENTION

1. Field of the Invention

The invention relates generally to the field of toxic polypeptides and their use in cancer chemotherapy.

2. Description of Related Art

Cancer, the uncontrolled growth of malignant cells, is a major health problem of the modern medical era and ranks second only to heart disease as a cause of death in the United States. Because almost all currently available antineoplastic agents have significant toxicities, such as bone marrow suppression, renal dysfunction, stomatitis, enteritis and hair loss, and because few agents are completely effective, new methods of treatment are needed.

Some malignancies, such as adenocarcinoma of the breast and lymphomas such as Hodgkin's Disease, respond relatively well to current chemotherapeutic, antineoplastic drug regimens. Chemotherapy is rarely curative in breast cancer. In a few malignancies, chemotherapy is curative in a high percentage of cases, e.g. acute lymphoblastic leukemia of childhood and Hodgkin's lymphoma, but not all such cases are cured, and other leukemias and lymphomas often are or become resistant to current chemotherapeutic drugs. In other cancers, some therapeutic value may be gained from chemotherapy, e.g. adenocarcinoma of the breast. Yet still other cancers respond poorly to chemotherapy, especially non-small cell lung cancer and pancreatic, prostate and colon cancers, and others. Even small cell cancer of the lung, initially chemotherapy sensitive, tends to return after remission, often with widespread metastatic spread leading to death of the patient.

### SUMMARY OF THE INVENTION

The present invention provides pharmaceutical compositions comprising synthetic polypeptides, and methods of using them for the treatment of cancer. The present invention provides an antimalignant agent which is significantly less toxic to non-malignant cells than to malignat cells, for use alone or in combination with anticancer drugs, surgery, or radiation to treat cancer.

The method for treating a mammalian subject having malignancy involves the step of administering to the subject a composition which comprises a sufficient amount of a polypeptide having the amino acid sequence of SEQ ID NO.: 1 (herein termed pDBD*4) to inhibit progress of the malignancy. Favored embodiments of the method involve administration of the polypeptide which has a terminus comprising a protein transfection domain.

The invention includes a polypeptide having the amino acid sequence of SEQ ID NO.:1. Embodiments include the polypeptide having a protein transfection domain.

### BRIEF DESCRIPTION OF THE FIGURES

FIG. 1 (A and B). Human leukemic ICR-27-4 cells were electroporated (500 μF, 300V) with 2 μg pDBD*4 peptide or without peptide (Control). Equal concentrations of cells were then plated and quantified for viable cell numbers over time.

FIG. 2. Effect of pDBD*4R9 on MCF-7 (breast cancer) and SWB-39 (glioma cells).

FIG. 3. Effect of peptide pDBD*4R9 on GT-MCF-7 cells, an independent strain of MCF-7 breast cancer cells.

FIG. 4. Effect of peptide pDBD*4R9 on human leukemic CEM C714 cells.

### DETAILED DESCRIPTION OF THE INVENTION

General Description and Definitions

The practice of the present invention will employ, unless otherwise indicated, conventional techniques within the skill of the art in (1) culturing animal cells, microorganisms, viruses, and bacteriophage; (2) biochemistry; (4) molecular biology; (5) microbiology; (6) genetics; (7) chemistry. Such techniques are explained fully in the literature. See, e.g. Culture of Animal Cells: A Manual of Basic Technique, 4th edition, 2000, R. Ian Freshney, Wiley Liss Publishing; Animal Cell Culture, eds. J. W. Pollard and John M. Walker; Plant tissue Culture: Theory and Practice, 1983, Elsevier Press; Maniatis et al., Molecular Cloning: A Laboratory Manual; Molecular Biology of The Cell, Bruce Alberts, et. al., 4th edition, 2002, Garland Science: Microbial Biotechnology, Fundamentals of Applied Microbiology, Alexander N. Glazer and Hiroshi Nikaido 1995, W.H. Freeman Co.; Pharmaceutical Biotechnology, eds. Daan J. A. Crommelin and Robert D. Sindelar, 1997, Harwood Academic Publishers; “Manual of Clinical Laboratory Immunology, eds. Noel R. Rose et al, 4th Edition, 1997, American Society for Microbiology); Molecular Cloning, eds. J. Sambrook, EF Fritsch & T. Maniatis, 1989, Cold Spring Harbor Laboratory Press

The method of the invention involves administering to cells in culture or to a mammalian subject carrying a malignancy a composition which comprises an effective amount of a polypeptide having the amino acid sequence of SEQ ID NO.: 1 to inhibit progress of the malignancy and/or kill the malignant cells. A preferred embodiment of the polypeptide includes a protein transfection domain, as described below, covalently attached to the N-terminus. The method is used for treating a wide variety of malignancies, including, but not limited to:
carcinoma, including that of the head and neck, bladder, breast, colon, kidney, liver, lung, ovary, pancreas, stomach, cervix, thyroid and skin, including squamous and basal cell carcinoma, and other dermal malignancies;
hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, Burketts lymphoma and multiple myeloma;
tumors of mesenchymal origin, including fibrosarcoma, rhabdomyoscarcoma, and osteosarcoma
other tumors, including melanoma, seminoma, teratocarcinoma, neuroblastoma and glioma;
tumors of the central and peripheral nervous system, including astrocytoma, neuroblastoma, and schwannomas; and
other tumors, including, xeroderma pigmentosum, keratoacanthoma, seminoma, thyroid follicular cancer and teratocarcinoma.
   Peptide pDBD*4

The synthetic polypeptide used in the invention is referred to herein as pDBD*4, which is a 32-amino acid polypeptide having the following sequence (from N-to C-terminus):

SEQ ID NO. 1

tyr-leu-cys-ala-gly-arg-asn-asp-cys-ile
ile-ala-ile-lys-phe-glu-glu-lys-thr-ala
gln-his-ala-ala-iso-glu-asn-val-phe-arg
leu glu

pDBD*4 kills a variety of kinds of cancer cells when introduced intracellularly. Treatment of individuals with pDBD*4 forms the basis for anti-cancer therapy of subjects having malignancies. The method involves the step of administering to the subject a composition which comprises an effective amount of a polypeptide having the pDBD*4 amino acid sequence.

pDBD*4 is a portion of a larger 89 aa peptide encoded by the gene 398-465*, which is a subject of U.S. Pat. No. 5,571,791, incorporated by reference. Also see Ji, Y., Johnson, B. H., Webb, M. S. Thompson, E. B. Mutational analysis of DBD*, a unique anti-leukemic gene sequence. Neoplasia 4:417-423; 2002.

The inventors herein have demonstrated that pDBD*4 causes cell death when it is transfected by electroporation into human lymphoblastic clonal cell lines ICR-27, CEM 1-15 and CEM C7-14 (derived from CEM lymphoblastic cells). Derivation of ICR-27 cells is found in Molecular & Cellular Biology (1981) 1:512-21 and of CEM1-15 and CEM7-14 in Genomics (2003) 81:175-97.

Adding a covalent sequence of polyarginine (arg or R)₉ to the N-terminus of pDBD*4 resulted in pDBD*R9, which spontaneously enters many kinds of cells.

As shown below, pDBD*R9 was administered to CEM cell clones ICR-27, C7-14, and C1-15. Two of these clones are resistant to the synthetic glucocorticoid dexamethasone, commonly used in the treatment of acute lymphoblastic leukemia. All were killed in a dose-dependent manner. Killing activity was also documented against SWB-39 glioma, MCF-7 breast cancer cells and basal cell carcinoma cells.

The results presented herein demonstrate that pDBD*4 or pDBD*R9 has killing activity against a wide variety of tumor types.

pDBD*4 was synthesized by the Peptide Synthesis Core of the UTMB Biomolecular Resources Facility, using solid state synthesis and 9-fluorenylmethoxycarbonyl (Fmoc) chemistry on ABI 430, 433 synthesizers. The Core uses HPLC and MALDI mass spectroscopy to verify purity of synthesized peptides. Synthetic methods for producing the subject peptides are well-known in the art. General means for the production of peptides, analogs or derivatives are outlined in Chemistry and Biochemistry of Amino Acids, Peptides and Proteins, A Survey of Recent Developments, Weinstein, B. ed., Marcell Dekker, Inc., publ. New York (1983). A wide variety of well-established techniques are available for synthesizing peptides (see Merrifield, B. Methods in Enzymology (1997) 289:3-13; Fields, GB and Noble, RL. Int. J. of Peptide & Protein Res. (1990) 35(3):161-214)).

pDBD*4 was tested in cell clones from the CEM leukemic cell system. Some of those clones are susceptible to apoptotic cell death evoked by steroids of the glucocorticoid class; others are not. Both types of clones were killed by electroporation of pDBD*4 into the cells, whereas exogenous application of pDBD*4 produced no effect.

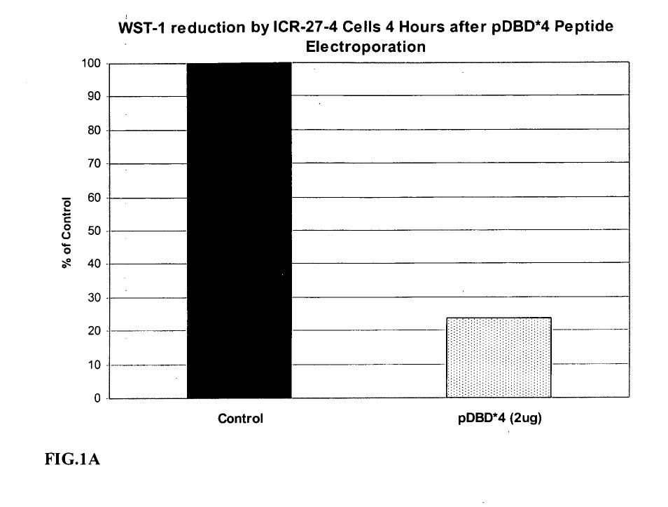

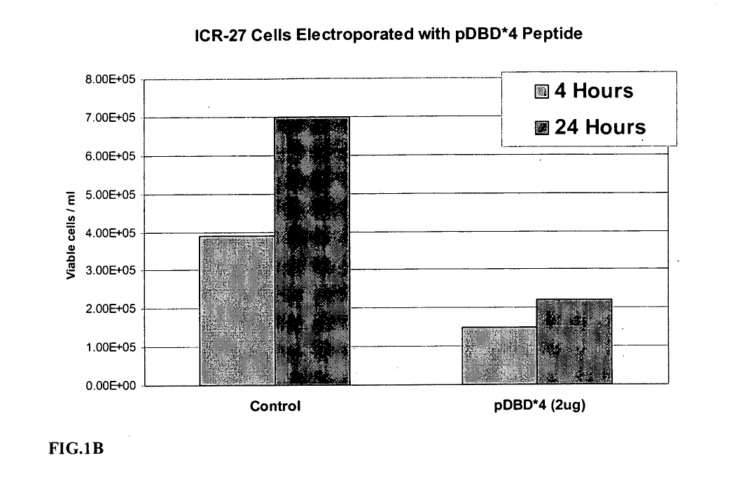

FIG. 1 shows an example of the cell-killing effect of electroporating pDBD*4 into the glucocorticoid-resistant clone ICR27-4.

We have previously established that the electroporation efficiency of these cells is about 40% (Nazareth, LV, et al. (1996) Leukemia, 10: 1789-95). The extent of cell kill by pDBD*4 seen in FIG. 1 is consistent with a highly effective agent being delivered to only a proportion of the cells, because of the delivery method.

To provide a more efficient method of delivery, a protein transfection domain was added to pDBD*4. In particular, nine-arginine (R9) sequence was added covalently to its N-terminus to make pDBD*4R9. Methods for making Protein transfection domains (PTDs) are well known in the art (Matsushita, M., Tomizawa, K., Moriwaki, A., Li, S. T., Terada, H. and Matsui, H. A high-efficiency protein transduction system demonstrating the role of PKA in long-lasting long-term potentiation. The Journal of Neuroscience 21(16):6000-6007, 2001; Rothbard, J. B., Garlington, S., Lin, Q., Kirschberg, T., Kreider, E., McGrane, P. L., Wender, P. A., and Khavari, P. A. Conjugation of arginine oligomers to cyclosporin A facilitates topical delivery and inhibition of inflammation. Nature Medicine 6(11):1253-1257, 2000; Chen, L., Wright, L. R., Chen, C. H., Oliver, S. F., Wender, P. A. Mochly-Rosen, D. Molecular transporters for peptides: delivery of a cardioprotective-PKC agonist peptide into cells and intact ischemic heart using a transport system, R7. Chemistry & Biology 8:1123-1129, 2001; Han, K., Jeon, M. J., Kim, S. H., Ki, D., Bahn, J. H., Lee, K. S., Park, J., and Choi, S. Y. Efficient intracellular delivery of an exogenous protein GFP with genetically fused basic oligopeptides. Molecules and Cells 12(2):267-271, 2001; Jin, L. H., Bahn, J. H., Eum, W. S., Kwon, H. Y., Jang, S. H., Han, K. H., Kang, T. C., Won, M. H., Kang, J. H., Cho, S. W., Park, J. and Choi, S. Y. Transduction of human catalase mediated by an HIV-1 tat protein basic domain and arginine-rich peptides into mammalian cells. Free Radical Biology & Medicine 31(11):1509-1519, 2001; Mitchell, D. J., Kim, D. T., Steinman, L., Fathman, C. G., Rothbard, J. B. Polyarginine enters cells more efficiently than other polycationic homopolymers. J. Peptide Res. 56:318-325, 2000).

Exposure of human leukemic clone ICR27-4 cells to increasing concentrations of pDBD*4R9 showed that it caused cell loss in a dose-dependent manner. At the maximum dose tested in this experiment, pDBD*4R9 caused over a 2-log loss of cells. A control, unrelated peptide containing a polyarginine PTD [GnRHR7] produced only slight growth inhibition, not dose-dependent (Table 1).

It should be understood that embodiments of pDBD*4 have an associated protein transfection domain (PTD). While a polyarginine transfection domain is exemplified herein, the compositions and methods of the invention employ pDBD*4 associated with any transfection domain (PTD), referred to herein as pDBD*4PTD provided the PTD permits the polypeptide to readily enter the target malignant cells of interest.

### EXAMPLE 1

TABLE 1
Reduction in ICR 27 leukemic cells by application of pDBD * 4R9
A. Cells/ml after 23 h

| | Vehicle Only | GnRHR7 | pDBD * 4R9 |
|---|---|---|---|
| | 6.4 × 1⁵ | | |
| 5 μM | | 5.1 × 10⁵ | 1.8 × 10⁵ |
| 10 μM | | 5.2 × 10⁵ | 1.95 × 10⁵ |
| 20 μM | | 5.3 × 10⁵ | 0.5 × 10⁵ |

At t=0, growing cells were removed from medium, washed, and plated at 4.0×10⁶ cells/ml in serum-free medium to which the peptide was added from a stock solution. Control peptide was GnRHR7 (Gonadotropin Releasing Hormone with a 7-arginine polymer added covalently). One hour later cells were diluted into growth medium. After 23 h, each culture was counted in duplicate. Average values are shown. Similar dose-related responses to pDBD*4R9 have been seen in 3 experiments.

Comparison of the ability of pDBD*4 and pDBD*4R9 to kill ICR-27 leukemic cells showed that without electroporation, pDBD*4R9 added exogenously killed the cells in a dose dependent manner, whereas pDBD*4 did not.

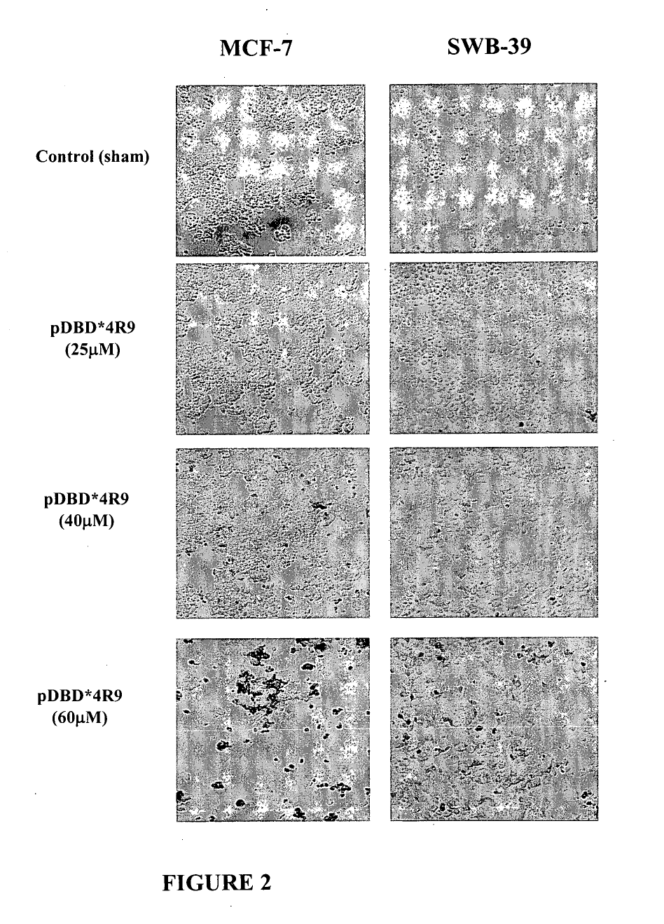

The use of a PTD in the embodiment pDBD*4R9 allowed tests of the effect of the invention on malignant cell types other than leukemic. Addition of pDBD*4R9 caused death of MCF-7 (breast cancer), SWB-39 (glioma) and a line of basal cell carcinoma cells. Examples of data from the former two cell lines are shown in FIG. 2.

A more quantitative assay was carried out using as an endpoint the reduction of a water-soluble tetrazolium dye (WST-1). This method has been employed to follow the effects of various chemotherapeutic agents on malignant cells (Hwang, W. S., Chen, L. M., Huang, S. H., Wang, C. C., Tseng, M. T. Prediction of chemotherapy response in human leukemia using in vitro chemosensitivity test. Leuk Res. 17:685-688, 1993; Pieters, R., Loonen, A. H., Huismans, D. R., Broekema, G. J., Dirven, M. W., Heyenbrok, M. W., Hahlen, K., Veerman, A. J. In vitro drug sensitivity of cells with children with leukemia using the MTT assay with approved culture conditions. Blood 76:2327-2336, 1990).

Viable cells can reduce the dye; dead cells cannot. The effects on pDBD*4R9 on MCF-7 breast cancer cells and non-malignant NIH-3T3 cells were compared (Table 2).

TABLE 2
WST-1 Absorbance, 450-595 nm.

| | Well 1 | Well 2 | Well 3 | Avg. |
|---|---|---|---|---|
| NIH-3T3 | | | | |
| Control | 0.252 | 0.222 | 0.21 | 0.228 |
| Frag4R9 | 0.199 | 0.216 | 0.196 | 0.204 |
| MCF-7 | | | | |
| Control | 0.417 | 0.506 | 0.544 | 0.489 |
| Frag4R9 | 0.045 | 0.087 | 0.289 | 0.140 |

20,000 cells were plated in triplicate in a 96 well plate. 24 hr later, cells were washed once and growth medium was replaced with serum free medium containing pDBD*4R9 (termed ‘Frag49’ in the table) or no peptide (Control) and placed in a tissue culture incubator at 37 deg. After 1 hr, serum-containing medium was added to each well and the plates were reincubated for 5 hr. 20 μl of WST-1 dye was then added to each well, and 4 hr later, Absorbance at 450-595 nm was determined spectrophotometrically. A chemical blank (avg. of 4 wells, medium plus dye only) was subtracted from each raw score.

Comparing the malignant non-malignant cells, the results show a favorable balance of effects. At this dose of pDBD*4R9, death of MCF-7 cells is seen by an average reduction of over 3-fold in their ability to metabolize the tetrazolium dye, whereas NIH-3T3 cells' metabolism of the dye was not significantly altered.

As shown in FIG. 1, ICR-27-4 cells were electroporated (500 μF, 300V) with 2 μg pDBD*4 peptide or without peptide (Control). Equal concentrations of cells were then plated.

In FIG. 1A, cell viability was evaluated after 4 hr using the WST-1 dye reduction assay (8,9). Quadruplicate wells of a 96 well plate each received 200 μl of cells plus 20 μl of WST-1 dye. The plate was then placed in a tissue culture incubator for 4 hours. Spectrophotometric analysis was carried out on a plate reader with Absorbance read at 450-595 nm. Absorbancies were corrected for the average of triplicate chemical blanks (200 μl growth medium plus 20 μl WST-1 dye). Data are presented as percent of control.

In FIG. 1B, cell concentrations were determined after 4 hr. and 24 hr. by hemocytometer counts of duplicate aliquots, with trypan blue dye added in the 24 hr samples to discriminate non-viable, but membrane-intact cells.

### EXAMPLE 2

This example shows the effect of pDBD*4R9 on the human leukemic cell line, CEM; human breast cancer line MCF-7; and colon cancer line, CaCo-2.

For each cell line, the protocol was the same for determining the effects of pDBD*4R9.

Cells were plated in equal numbers and washed free of growth medium.

The peptide pDBD*4R9 was then added to the cells for one hour, after which they were returned to growth medium.

At a suitable interval thereafter, they were assayed for viability by use of the WST assay (described above), which measures the ability of viable cells' mitochondria to metabolize and therefore change the color of a water soluble tetrazolium dye (WST). By measuring the absorbance of the color, one has a quantitative correlate of viability: More absorbance=more viable cells.

In the experiment on MCF-7 cells, an equivalent assay (AlarmarBlue) was used.

The data for the colon cancer cell line CaCO was as follows.

| | Absorbance | (viability) | |
|---|---|---|---|
| Expt 1. | | | |
| control cells | 0.33 +/− 0.01 | (100%) | triplicate samples, |
| pDBD * 4R9 | | | avg +/− 1SD |
| 25 microM | 0.08 +/− 0.01 | (24%) | duplicates, avg +/− range |
| 50 | 0.16 +/− 0.005 | (48%) | |
| Expt 2. | | | |
| control | 0.41 +/− 0.06 | (100%) | quintuplicates, avg +/− 1SD |
| 100 microM | 0.19 +/− 0.05 | (46%) | triplicates, avg +/− 1SD |

These data show a significant anticancer effect on colon cancer cells, with 50-75% estimated cell kill after a single treatment with the peptide. The same range of cell kill is seen at all three concentrations, which may indicate maximum effect in these cells for a single treatment.

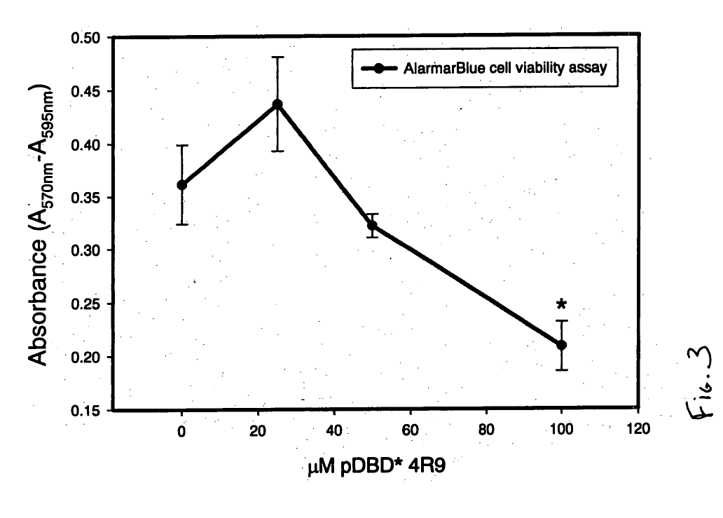

FIG. 3 demonstrates the effects of a four-hour pDBD*4R9 treatment on breast cancer GT-MCF-7 cells.

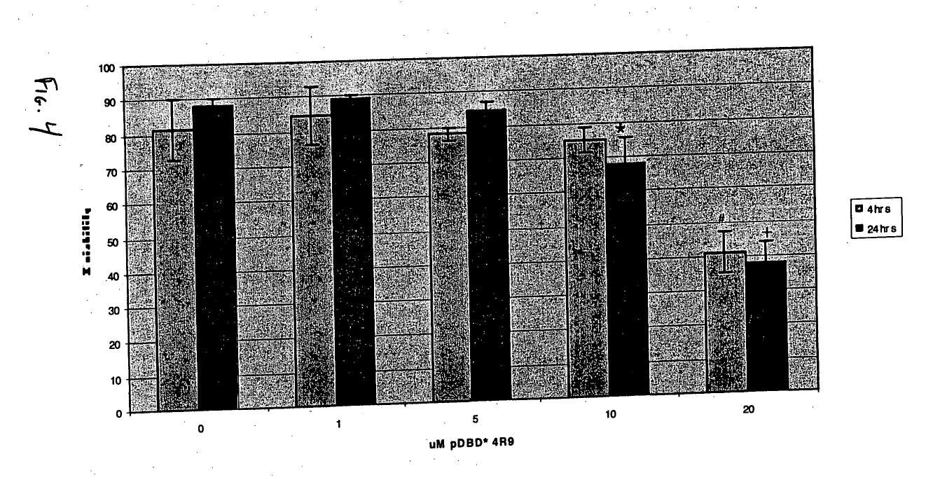

FIG. 4 demonstrates the effects of pDBD*4R9 treatment on human leukemic CEM C714 cells. FIGS. 3 and 4 demonstrate significant anticancer effects on breast cancer GT-MCF-7 cells and on human leukemic CEM C714 cells.

Treating Patients—Administration of pDBD*4PTD

The compositions of the present invention may be formulated in a pharmaceutical composition, which may include carriers, thickeners, diluents, buffers, preservatives, surface active agents, liposomes, or lipid formulations, and the like. The pharmaceutical compositions may also include one or more additional active ingredients such as other chemotherapy agents, antimicrobial agents, antiinflammatory agents, anesthetics, and the like.

The pharmaceutical composition may be administered in a number of ways depending on whether local or systemic treatment is desired, and on the area to be treated. Administration may be topically (including on the skin, ophthalmically, vaginally, rectally, intranasally), orally, by inhalation, or parenterally, for example by intravenous, subcutaneous, intratumor, intraperitoneal, or intramuscular injection.

Formulations for topical administration may include ointments, lotions, creams, gels, drops, suppositories, sprays, liquids and powders. Conventional pharmaceutical carriers, aqueous, powder or oily bases, thickeners, and the like may be necessary or desirable. Compositions for oral administration include powders or granules, suspensions or solutions in water or nonaqueous media, capsules, or tablets. Thickeners, flavorings, diluents, emulsifiers, dispersing aids or binders may be desirable. Formulations for parenteral administration may include sterile aqueous solutions optionally containing buffers, liposomes, diluents and other suitable additives (Remington's Pharmaceutical Science, Mack Publishing Co, NJ (1991); Goodman & Gilman's The Pharmacological Basis of Therapeutics, 9.sup.th Ed., 1996, McGraw-Hill, esp. Chabner et al., Antineoplastic Agents) otherwise known in the art.

Dosing is dependent on the severity and responsiveness of the condition to be treated, with course of treatment lasting from several days to several months or until a cure is effected or a diminution of disease state is achieved, or an adverse reaction (e.g. allergy to the peptide) occurs. However the small size of the peptide and its delivery without immunological adjuvant should delay or minimize its antigenicity (Essential Immunology, 6^{th} ed. Ivan M. Roitt. Blackwell Scientific Publications, Oxford. Principles of Bacteriology, Virology and Immunity, 6^{th} ed. GS Wilson and A Miles. 1975. Williams & Wilkins, Baltimore. Vol I, pp329+).

It should be understood that the dosage ranges set forth below are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can also be adjusted as the various factors change over time.

Determination of the proper dosage for a particular situation is within the skill of the art. In general, routine experimentation in preclinical followed by clinical trials will determine specific ranges for optimal therapeutic effect, for each therapeutic, each administrative protocol, and administration to specific patients will also be adjusted to within effective and safe ranges depending on the patient condition and responsiveness to initial administrations. However, the ultimate administration protocol will be regulated according to the judgment of the attending clinician considering such factors as age, condition and size of the patient

In clinical trials, treatment is initiated with smaller dosages which are less than the optimum dose of the compound. Thereafter, the dosage is increased by small amounts, reaching a fully therapeutic dose, which results in the optimum effect under the circumstances. In actual practice, the fully therapeutic dose, as determined in clinical trials, is administered.

The therapeutics of the invention can be administered in a therapeutically effective dosage and amount, in the process of a therapeutically effective protocol for treatment of the patient. The initial and any subsequent dosages administered will depend upon the patient's age, weight, condition, and the disease, disorder or biological condition being treated. Depending on the therapeutic, the dosage and protocol for administration will vary, and the dosage will also depend on the method of administration selected, for example, local or systemic administration.

Combination Therapy

In accordance with another aspect of this invention, the method of this invention can be used in combination with a conventional cancer chemotherapy, with the result that the treatment with pDBD*4PTD will increase the sensitivity of the tumor to conventional cancer chemotherapy and result in greater effectiveness of the conventional cancer chemotherapy drug. For example, the method of this invention can be complemented by a conventional radiation therapy or chemotherapy. Thus, in one embodiment of this invention, the method of this invention comprises administering to a patient a composition comprising pDBD*4PTD and another anticancer agent. Any anticancer agent known in the art can be used in this invention so long as it is pharmaceutically compatible with pDBD*4PTD.

By “pharmaceutically compatible” it is intended that the other anticancer agent will not interact or react with the above composition, directly or indirectly, in such a way as to adversely affect the effect of the treatment of cancer, or to cause any significant adverse side reaction in the patient.

Exemplary anticancer agents known in the art include busulphan, chlorambucil, hydroxyurea, ifosfamide, mitomycin, mitotane, chlorambucil, mechlorethamine, carmustine, lomustine, cisplatin, carmustine, herceptin, carboplatin, cyclophosphamide, nitrosoureas, fotemustine, vindescine, etoposide, daunorubicin, adriamycin, paclitaxel, docetaxel, streptozocin, dactinomycin, doxorubicin, idarubicin, plicamycin, pentostatin, mitotoxantrone, valrubicin, cytarabine, fludarabine, floxuridine, clardribine, methotrexate, mercaptopurine, thioguanine, capecitabine, irinotecan, dacarbazine, asparaginase, gemcitabine, altretamine, topotecan, procarbazine, vinorelbine, pegaspargase, vincristine, rituxan, vinblastine, tretinoin, teniposide, fluorouracil, melphalan, bleomycin, salicylates, aspirin, piroxicam, ibuprofen, indomethacin, naprosyn, diclofenac, tolmetin, ketoprofen, nambuetone, oxaprozin, doxirubicin, nonselective cycclooxygenase inhibitors such as nonsteroidal anti-inflammatory agents (NSAIDS), and selective cyclooxygenase-2 (COX-2) inhibitors.

The anticancer agent used can be administered simultaneously in the same pharmaceutical preparation with the pDBD*4PTD. The anticancer agent can also be administered at about the same time but by a separate administration. Alternatively, the anticancer agent can be administered at a different time from the administration of the pDBD*4PTD. Some minor degree of experimentation may be required to determine the best manner of administration, this being well within the capability of one-skilled in the art once apprised of the present disclosure.

The methods of this invention are particularly useful in treating humans. Also, the methods of this invention are suitable for treating cancers in animals, especially mammals such as canine, bovine, porcine, and other animals.

pDBD*4PTD may be used as an adjuvant following surgery to remove a tumor. It may be then administered locally to attack remaining tumor cells possibly missed in the region of the surgical wound. Alternatively, it may be administered systemically or regionally to reach tumor cells beyond the reach of surgical approaches or in loci where surgery would endanger vital structures.

Antiproliferative Assays

Assays for measuring the inhibitory effects of the peptides of the invention and methods of using them on the progress of malignancy, including diminished proliferation or diminished tumor growth, tumor regression or tumor disappearance i.e. determining anti-malignant or anti-tumor activity in vivo are well known in the art (e.g. MRI, CAT scans, radiograms, physical exams, biochemical tests, blood cell evaluations, among others, are used to follow and evaluate patients during treatment. As to in vitro assays, non-limiting examples are found in El-Naghy, M., et al., Exp. Cell Research (2001) 270:166-75; Miller, AL, et al., Neoplasia (2002) 4:69-81; and Thompson, EB. Ann. Rev. of Physiol (1998) 60:525-32, all incorporated by reference.

What is claimed is:

1. A method for treating a mammalian subject having malignancy, said method comprising the step of administering to said subject a composition which comprises a sufficient amount of a polypeptide having the amino acid sequence of SEQ ID NO.: 1 to inhibit progress of said malignancy in said subject.

2. The method of claim 1 wherein a terminus of said polypeptide further comprises a protein transfection domain.

3. The method of claim 1 wherein said protein transfection domain is polyarginine.

4. The method of claim 1 wherein said malignancy is selected from the group of malignancies consisting of melanoma, colorectal cancer, prostate cancer, breast cancer, ovarian cancer, cervical cancer, endometrial cancer, glioblastoma, renal cancer, bladder cancer, gastric cancer, pancreatic cancer, neuroblastoma, lung cancer, leukemia and lymphoma, basal cell carcinoma, dermal malignancies, head and neck cancers.

5. The method of claim 1 in combination with anti-cancer therapy selected from one or more of the group consisting of chemotherapy, surgery, and radiation.

6. A polypeptide having the amino acid sequence of SEQ ID NO.:1.

7. The polypeptide of claim 6 further comprising a terminally situated protein transfection domain.

8. The polypeptide of claim 7 wherein said protein transfection domain is polyarginine.

9. A pharmaceutical composition comprising the polypeptide selected from one or both of the group consisting of the polypeptide having amino acid sequence of SEQ ID NO.: 1, and the polypeptide of SEQ ID NO.: 1 comprising a terminally situated protein transfection domain.
